(19) United States
(12) Patent Application Publication
(10) Pub. No.: US 2023/0063918 A1
(43) Pub. Date: Mar. 2, 2023

(54) DEVICE FOR DELIVERING MEDICATION WITH INTEGRATED INTERPOSER AND MICROPUMP

(71) Applicant: AITA BIO INC., New York, NY (US)

(72) Inventors: Peter Smeys, San Jose, CA (US); Andrei Papou, San Jose, CA (US); James Lew, San Leandro, CA (US)

(21) Appl. No.: 17/792,137

(22) PCT Filed: Jan. 19, 2021
(86) PCT No.: PCT/US21/13961

§ 371 Date: Jul. 11, 2022

Related U.S. Application Data
(60) Provisional application No. 62/963,417, filed on Jan. 20, 2020.

(51) Int. Cl. A61M 5/172; A61M 5/142; A61M 5/158

(52) U.S. Cl. CPC A61M 5/1723; A61M 5/14244; A61M 5/158; A61M 2205/3331; A61M 2205/8206; A61M 2230/201

(57) ABSTRACT

An interposer to be used in a device for delivering medication to a patient is disclosed. The device includes a reservoir for storing the medication and a needle for releasing the medication in the patient, the interposer configured to mount the reservoir and needle. The interposer comprises a channel for distributing the medication from the reservoir to the needle, a thin membrane defining a portion of the channel as a chamber for receiving the medication, and a piezoelectric transducer positioned on the thin membrane that functions as an actuator for moving the thin membrane toward and away from the chamber of the channel.

### REFERENCE TO RELATED APPLICATIONS

This application claims priority to U.S. provisional application No. 62/963,417, filed on Jan. 20, 2020 entitled “Device For Delivering Insulin With Integrated Interposer and Micropump,” which is incorporated by reference herein.

### FIELD OF THE INVENTION

The present invention relates to a device for delivering insulin with integrated interposer and micropump.

### BACKGROUND OF THE INVENTION

Various infusion systems exist that utilize devices for delivering liquid medication or other therapeutic fluid to patients (users) subcutaneously. For patients with diabetes mellitus, for example, conventional infusion systems incorporate various pumps that are used to deliver insulin to a patient. These pumps have the capability of delivering assorted fluid delivery profiles which include specified basal rates and bolus requirements. For example, these pumps include a reservoir to contain the liquid medication along with electromechanical pumping technology to deliver the liquid medication via tubing to a needle that is inserted subcutaneously into the patient.

Although such conventional pumps/infusion systems are adequate for their intended purpose, such pumps have difficult controlling drug delivery precisely thereby causing harm to the patient. That is, these pumps have large stroke volumes resulting in inaccurate basal rate infusion and incorrect insulin dosing. Further, with these infusion systems, diabetes patients must install and carry at least two bulky and obtrusive devices on their bodies. This causes significant inconvenience for the patient during his/her daily activities.

Therefore, it would be advantageous to provide an improved infusion system over these conventional infusion systems.

### SUMMARY OF THE INVENTION

A device for delivering insulin with integrated interposer and micropump is disclosed.

In accordance with an embodiment of the present disclosure, a device configured as a fully autonomous and integrated wearable apparatus for managing delivery of a medication, the device comprising: a reservoir for storing the medication for subsequent delivery to a patient; a needle for delivering the medication to the patient subcutaneously; a micropump configured to pump the medication from the reservoir through the needle for delivering the medication to the patient; control circuitry controlling operations of a micropump; and an interposer integrated with the micropump, the interposer configured as an adapter for mounting the reservoir, the control circuitry and the needle, the interposer including a channel for distributing the medication from the reservoir to the needle and a thin membrane defining a portion of the channel as a chamber; wherein the micropump includes a piezoelectric transducer positioned on the thin membrane that functions as an actuator for deforming the thin membrane.

In accordance with another embodiment of the present disclosure, a device configured as a fully autonomous and integrated wearable apparatus for delivery management, the device comprising: a reservoir for storing the medication for subsequent delivery to a patient; a needle for delivering the medication to the patient subcutaneously; a MEMS device configured as a pump for pumping the medication from the reservoir through the needle or as a valve for preventing medication from flowing through device, the MEMS device including a piezoelectric transducer that functions as an actuator; control circuitry controlling operations of the piezoelectric transducer; and an interposer integrated with the piezoelectric transducer, the interposer configured as an adapter for mounting the reservoir, the control circuitry and the needle, the interposer including a channel for distributing the medication from the reservoir to the needle and a thin membrane defining a portion of the channel as a chamber, wherein the piezoelectric transducer is positioned on the thin membrane, thereby deforming the thin membrane as the piezoelectric transducer is actuated.

In accordance with another embodiment of the present disclosure, an interposer to be used in a device for delivering medication to a patient, the device including a reservoir for storing the medication and a needle for releasing the medication in the patient, the interposer configured to mount the reservoir and needle, the interposer comprising: a channel for distributing the medication from the reservoir to the needle; a thin membrane defining a portion of the channel as a chamber for receiving the medication; and a piezoelectric transducer positioned on the thin membrane that functions as an actuator for moving the thin membrane toward and away from the chamber of the channel.

### BRIEF DESCRIPTION OF DRAWINGS

FIG. 1 depicts a block diagram of the components of an example device for delivering insulin to a diabetes patient.

FIG. 2 depicts a top view of an example interposer shown in FIG. 1.

FIG. 3 depicts a cross sectional view of the example interposer in FIG. 2 along lines 3-3.

FIG. 4 depicts a perspective view of the assembly of certain components of the device in FIG. 1.

FIG. 5A depicts another perspective view of the assembly of components of the device in FIG. 1.

FIG. 5B depicts an exploded view of the assembly of components of the device in FIG. 1.

FIG. 6 depicts a cross sectional view of another example interposer with an integrated micropump.

FIG. 7 depicts a top view of another example interposer with an integrated micropump.

### DETAILED DESCRIPTION OF THE INVENTION

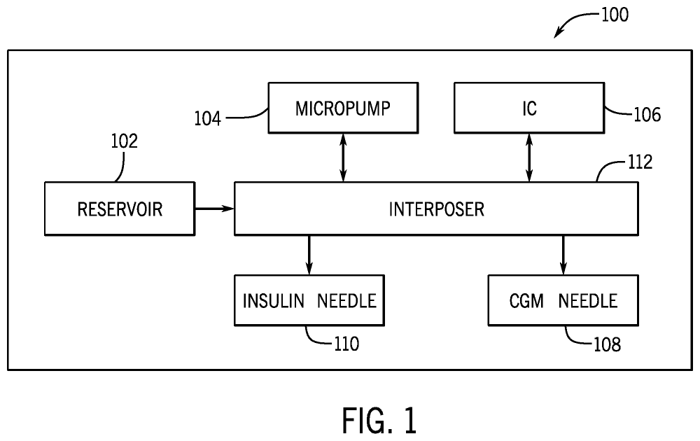

FIG. 1 depicts a block diagram of the salient components of device 100 for delivering insulin (or other medication or fluid) to a diabetes patient (also referred to as a user of device 100). Device 100 is configured as a fully autonomous and integrated wearable apparatus for diabetes management in which continuous glucose monitoring (CGM), insulin delivery and control functionality are provided together to ensure insulin is delivered at very precise rates. Device 100 includes several components or modules including, among other components, reservoir 102, micropump 104 (also referred to as small pump or pump), control circuitry (integrated circuit—IC) 106, insulin needle 108, continuous glucose monitoring (GCM) or analyte sensing needle 110 and interposer 112. Device 100 also includes a battery (not shown) that provides power to IC 106 and micropump 104 (battery may be on a printed circuit board (PCB) as described below). Micropump 104 includes one or more MEMS devices (micro-electro-mechanical systems devices, i.e., piezoelectric transducer), as known to those skilled in the art, into its architecture for pump and/or valve functionality and/or flow/pressure sensing as discussed below. (MEMS device with valve functionality may also be referred to as a microvalve or valve). However, micropump 104 may incorporate other pumping mechanisms to achieve desired results as known to those skilled in the art. (Besides inulin, device 100 may be configured to infuse other medications such as small molecule pharmaceutical solutions, large molecule or protein drug solutions, saline solutions, blood or other fluids known to those skilled in the art.)

Reservoir 102 is configured to store insulin for subsequent delivery to the patient via insulin needle 108 as known to those skilled in the art. Micropump 104 is configured to pump insulin through insulin needle 108 (releasing the insulin) into the patient. Control circuitry 106, as known to those skilled, in the art is configured to control the operation of the micropump 104. CGM or analyte sensor needle 110 is configured to monitor glucose levels in the patients and transmit the data to control circuitry 106.

Interposer 112 is configured as an adapter for (1) mounting reservoir 102, micropump 104, control circuitry (integrated circuit—IC) 106, insulin needle 108 and CGM needle 110 and for (2) redistributing fluid through channels and electrical signals between those components. That is, interposer 112 functions to fully integrate reservoir 102, micropump 104, control circuitry (IC) 106, insulin needle 108 and GCM or analyte sensor needle 110 in order to reduce the amount of tubing and wiring to connect such components and miniaturize the delivery device. Interposer 112 also includes a flow sensor or pressure sensor (not shown), e.g., as a separate MEMS device, to monitor flowrate of the insulin and/or occlusion of the pump as known to those skilled in the art. Interposer 112 is constructed of glass, but it may be made of other materials known to those skilled in the art. With interposer 112, the only connections or openings to the environment is a fill port, needle opening, sensor needle opening and connector pins for connecting power, ground and control signals to a printed circuit board (PCB). This is described in more detail below.

These components of device 100 are mounted to interposer 112 using laser, adhesive bonding, flip chip or other methods known to those skilled in the art. Electrical connections from micropump 104 to interposer 112 are made using wirebond or other means of connection known to those skilled in the art. Electronic connections from IC 106 to interposer 112 are constructed using wirebond, flip chip or other means known to those skilled in the art. Micropump 104 and integrated circuit (IC) 106 may be mounted at wafer level by die to wafer automated pick and place. Reservoir 102 and a spacer 114 (discussed below) may be mounted at wafer level or using other assembly processes known to those skilled in the art. Interposer fabrication is discussed below.

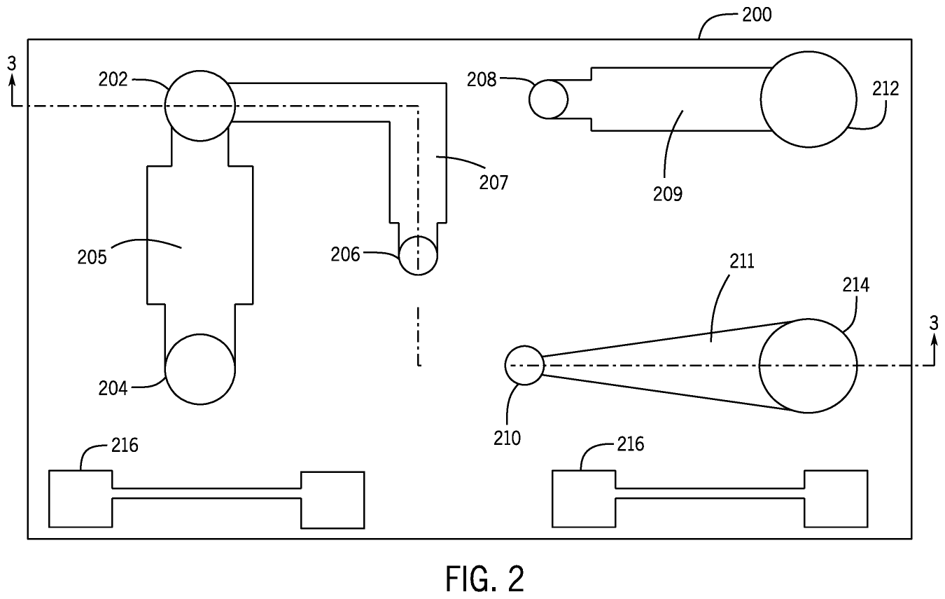

FIG. 2 depicts a top view of interposer 200 as identified in FIG. 1 (reference renumbered). In this figure, fluid channels, inlet and outlet ports and interconnects as shown are described below.

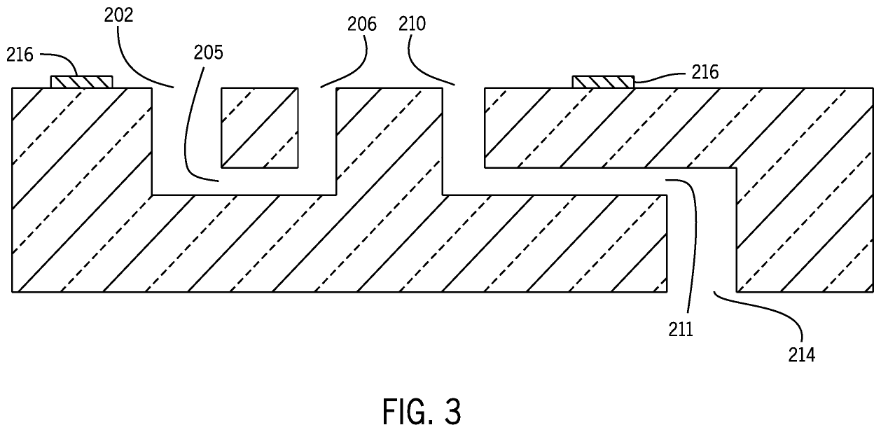

FIG. 3 depicts a cross sectional view of the interposer 200 in FIG. 2 along lines 3-3. This interposer is an example of an adapter that depicts (1) several ports that function as openings for fluid paths (e.g., channels) through the interposer for fluidly connecting reservoir 102, micropump 104, CGM needle 108 and insulin needle 110 and (2) interconnects for electrically connecting components IC 106, micropump 104 and CGM needle 108.

Specifically, interposer 200 includes top ports 202, 204 that are connected by channel 205 as shown as well as top port 206 that communicates with top port 202 via channel 207. Top port 202 may for example communicate with a port on micropump 104 and port 206 may communicate with reservoir 102. Top ports 208 communicates with bottom port 212 via channel 209 and port 210 communicates with bottom port 214 via channel 211. Top ports 208 and 210 may for example communicate with ports on micropump 104 and bottom ports 212 and 214 may communicate with insulin needle 106 and CGM or analyte sensor needle 108, respectfully (or cannulas).

Example measurement for top port 202 (of channel) may be 300 μm, top port 206 (channel) may be 100 μm and top port 210 may be 100-200 μm. Bottom port 212 may be 300 μm. The channel between top port 210 and bottom port 214 may vary but an example may be 25 μm. The height of the interposer 200 may be 800 μm for example. However, those skilled in the art know that the ports may be configured to various sizes/measurements to achieve desired effects. Interconnects 216 are also shown along with the ports in interposer 200. Interconnects 216 are configured as electrical connectors or conduits that enable the connection between micropump 104, CGM or analyte sensor 108 (and battery) and IC 106 as known to those skilled in the art. Interconnects may have thickness as 20 μm in narrow portion 90 μm as the ends. However, the interconnects may be any measurement to achieve desired results. In short, interposer 200 includes both ports/channels and interconnects to distribute fluid channels and electrical signals, respectively. (Interposer 200 may be constructed of a transparent material such as glass or any other transparent or non-transparent material known to those skilled in the art.)

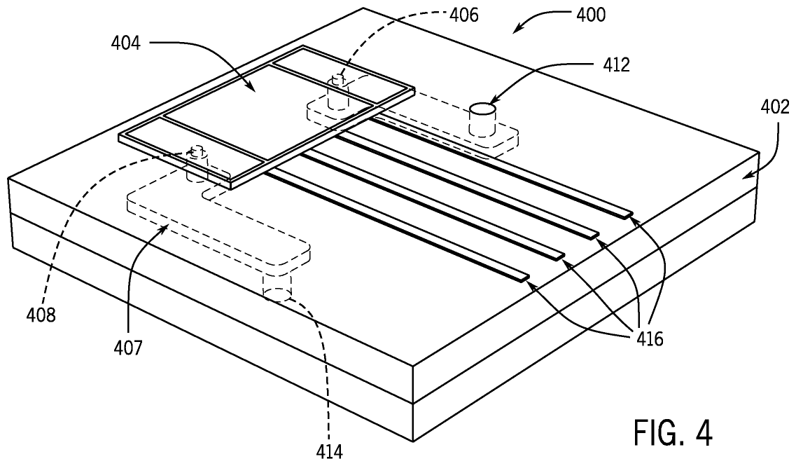

FIG. 4 depicts a perspective view of the assembly of certain components (including micropump) of an example device 400 for delivering insulin to a diabetes patient in FIG. 1 (reference renumbered). That is, FIG. 4 shows an assembly of components such as a micropump onto an interposer as described below.

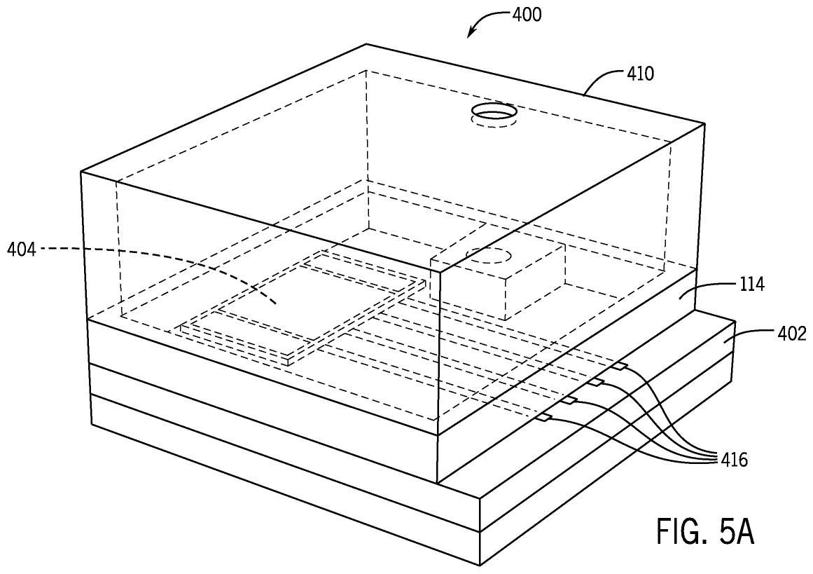

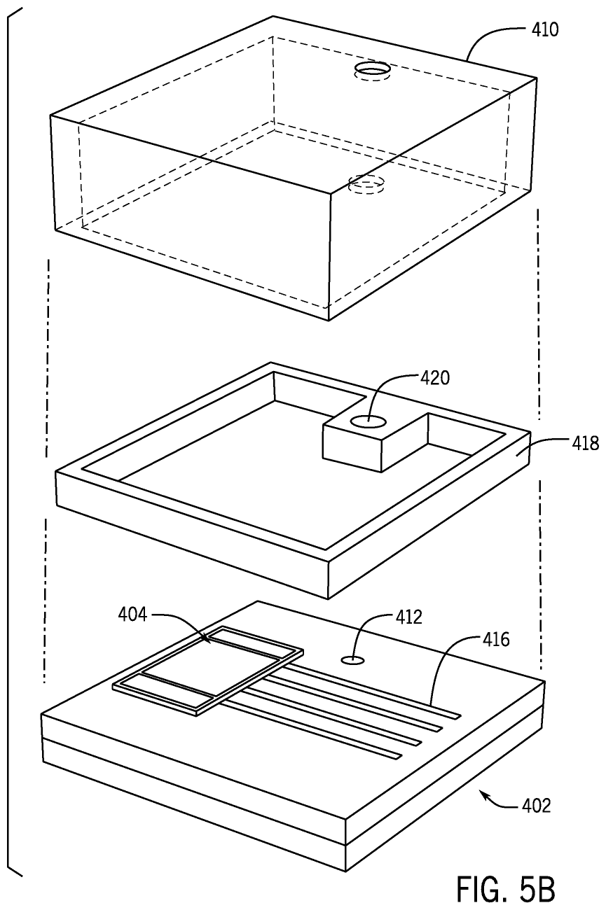

FIGS. 5A and 5B depict perspective views of the assembly of the example device 400 shown in FIG. 1. Example device 400 includes another example interposer 402 (different embodiment than interposer 200 in FIGS. 2 and 3). Device 400 includes micropump 404 (e.g., one or more MEMS devices) that is assembled on top of interposer 402 as shown and it communicates with ports 406, 408 to (1) withdraw insulin from reservoir 410 through inlet port 412 and (2) propel insulin through fluid channel 407 and out outlet port 414. As indicated above, (metal) interconnects 416 are used as connectors (i.e., traces) for connecting micropump 404 to the integrated circuit (IC), battery (not shown in FIGS. 4, 5A, 5B) as known to those skilled in the art. Device 400 also includes spacer 418 between reservoir 410 and interposer 402. Spacer 418 creates a standoff between reservoir 410 and interposer 402 to provide space for the micropump 404 and IC (not shown). Spacer 418 is configured to be of smaller size than interposer 402 to enable external connections as shown. Spacer 418 can be fabricated out of silicon, plastic or other material known to those skilled in the art. Spacer 418 has a through channel 420 that connects interposer 402 to reservoir 410 for drug delivery. That is, channel 420 is configured to enable flow from reservoir 410 into inlet port 412. (Interposer 402 may be constructed as a transparent material such as glass/Silicon or any other transparent or non-transparent material as known to those skilled in the art.)

The process for assembling the components onto an interposer is now described. The process proceeds to step 1, wherein a pump die is mounted onto the interposer using adhesive bonding (die-die or die wafer). (Steps not shown.) Next at step 2, control ICs are mounted onto the interposer using adhesive bonding (die-die or die-wafer). The process proceeds to step 3, wherein the pump and ICs are wirebonded down to the interposer. Next, a spacer is mounted onto the interposer using adhesive bonding at step 4. As indicated above, the spacer creates a standoff between a reservoir and interposer to provide space for the micropump and ICs. As indicated above, the spacer can be fabricated out of silicon, plastic or other material known to those skilled in the art. The spacer has a through hole that connects the interposer to the reservoir for drug delivery. The process proceeds to step 5, wherein the reservoir is mounted onto the spacer using adhesive bonding. The reservoir and spacer can be combined into a single component if desired.

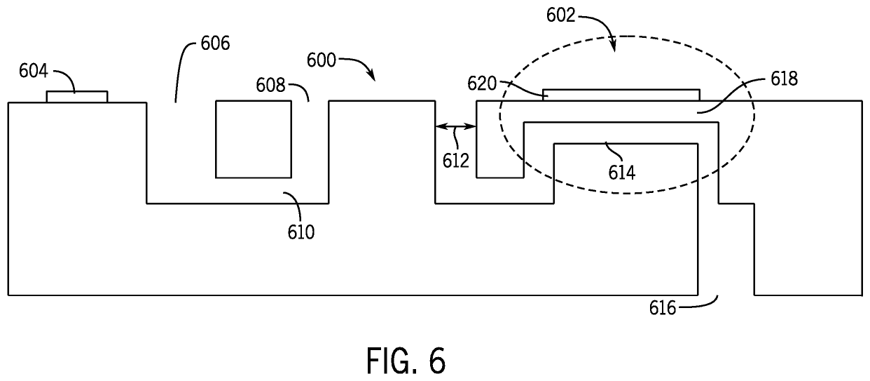

FIG. 6 depicts a cross sectional view of another example interposer 600 with an integrated micropump. Similar to the other examples disclosed herein, this interposer 600 is an example of another adapter that depicts (1) several ports that function as fluid paths through the interposer for fluidly connecting a reservoir (not shown), a micropump 602, CGM or analyte sensor needle and insulin needle (both not shown) and (2) interconnects 604 for electrically connecting components such as an integrated circuit (IC) (not shown), micropump 602 and a CGM or analyte needle. Specifically, the ports include top ports 606, 608 that are connected by channel 610 as shown as well as top port 612 that communicates with bottom port 616 via channel (i.e., chamber or cavity) 614. Channel 614 (or portion thereof) functions as a pumping or valving chamber as described in more detail below. The walls of interposer 600 (including the thin membrane described in detail below) define the shape of the channel 614 and the ports 606, 606, 612 and 616. If interposer is made of glass, then thin membrane 618 is a glass layer that will deform as piezoelectric transducer 620 is actuated as described below. Example measurements for the port 612 may be 100-200 μm, top port and bottom port may be 300 μm. The width of interposer 600 may be 500-5000 μm. However, those skilled in the art know that the ports may be configured to various measurements and sizes to achieve desired effects.

In this example interposer 600, however, interposer 600 and micropump 602 are integrated together (dotted circular line) as a single unit to form a pump, valve, pressure sensor or flow sensor as described below. (That is, interposer 600 may also be described as including or integrated with micropump 602 itself.)

Specifically, micropump 602 comprises thin membrane 618 (wall) of the interposer structure itself and piezoelectric transducer 620 (also referred to as a piezo) that is positioned on thin membrane 618. The thin membrane 618 defines a portion of the channel 614 as a chamber. Piezoelectric transducer 620 functions as an actuator for (1) a pump or valve or (2) a pressure or flow sensor as desired, thus eliminating the silicon layer(s) itself (of a MEMS device) as the pumping/valving/sensing component. As a pump, piezoelectric transducer 620 functions as an actuator that causes thin membrane 618 to bend/deflect or deform with respect to the channel/chamber 614, increasing or reducing pressure within channel or chamber 614, thereby displacing insulin from channel/chamber 614 or drawing insulin into channel/chamber 614 as known to those skilled in the art. As a valve, piezoelectric transducer 620, causes thin membrane 618 (of MEMS device) to bend/deflect or deform and close off the channel/chamber 614 preventing insulin flow through channel/chamber 614 entirely. As a sensor, pressure or flow within channel/chamber 614 is detected as insulin passes through it as known to those skilled in the art. In this example interposer 600, piezoelectric transducer 620 is integrated with the structure of interposer 600 itself to function as an actuator for a pump, valve or sensor. However, piezo 620 may be fabricated subsequent to interposer 600 construction. (Micropump or microvalve may be referred to as a microdevice or MEMS device as described above).

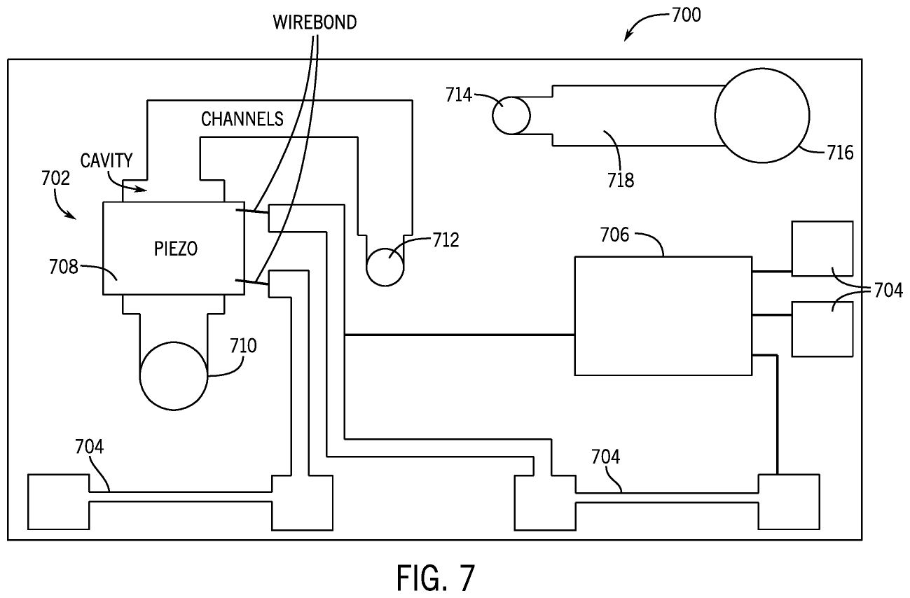

FIG. 7 depicts a top view of another interposer 700. This interposer is an example of another adapter that depicts (1) several ports that function as fluid paths through the interposer for fluidly connecting a reservoir (not shown), a micropump 702, CGM or analyte needle and insulin needle (both not shown) and (2) interconnect 704 for electrically connecting components including an integrated circuit (IC) 706, micropump 702 and CGM needle. Micropump 702 is constructed (and functions) similarly to the example shown in FIG. 6 wherein piezoelectric transducer 708 is integrated in interposer 700 itself on a thin membrane (not shown) and functions as an actuator for a pump or valve as described herein.

In this example, the interposer ports include top ports 710, 712 that are connected by channel as shown as well as top port 714 that communicates with bottom port 716 via channel 718. Top port 712 may measure 100 μm, bottom port 716 may measure 300 μm. Interconnect 704 may have width of 20 μm. These are only example measurements. However, those skilled in the art know that any number of ports (top or bottom) may be used and the ports may be configured to various sizes/measurements as known to those skilled in the art to achieve desired effects.

Similar to the examples described above, interposers in FIGS. 6 and 7 are fabricated of glass, but could be made of other materials as known to those skilled in the art. The fabrication process uses selective layer etching as known to those skilled in the art. The channels and cavities are created at various depths below the surface, thereby creating the thin membranes. The membranes may be as thin as 20 μm or less as known to those skilled in the art. The piezoelectric transducer is fabricated on the top surface of the interposer, over the membrane as shown. The piezoelectric transducer on the glass membrane now thus functions as a piezoelectric pump/valve or flow/pressure sensor, thus eliminating a separate silicon or other component. The piezoelectric transducer can be fabricated using the semiconductor process flow described above or using pick and place and wirebonding.

It is to be understood that the disclosure teaches examples of the illustrative embodiments and that many variations of the invention can easily be devised by those skilled in the art after reading this disclosure and that the scope of the present invention is to be determined by the claims below.

1. A device configured as a fully autonomous and integrated wearable apparatus for managing delivery of a medication, the device comprising:
a reservoir for storing the medication for subsequent delivery to a patient;
a needle for delivering the medication to the patient subcutaneously;
a micropump configured to pump the medication from the reservoir through the needle for delivering the medication to the patient;
control circuitry controlling operations of a micropump; and
an interposer integrated with the micropump, the interposer configured as an adapter for mounting the reservoir, the control circuitry and the needle, the interposer including a channel for distributing the medication from the reservoir to the needle and a thin membrane defining a portion of the channel as a chamber;
wherein the micropump includes a piezoelectric transducer positioned on the thin membrane that functions as an actuator for deforming the thin membrane.

2. The device of claim 1 wherein the micropump includes the thin film membrane that deforms in response to actuation of the piezoelectric transducer thereby increasing or decreasing pressure within the chamber of the channel.

3. The device of claim 1 wherein the interposer has one or more ports on a first side of the interposer and one or more ports on a second side of the interposer, the one or more ports on the first side communicate with the one or more ports on the second side via the channel.

4. The device of claim 1 wherein the micropump includes one or more MEMS devices with pump and/or valve functionality.

5. The device of claim 1 wherein the micropump comprise a flow sensor or pressure sensor to monitor flowrate of the medication and/or occlusion of the micropump.

6. The device of claim 1 further comprising a plurality of interconnects electrically connecting the control circuitry and micropump.

7. The device of claim 1 wherein the medication is insulin.

8. The device of claim 6 wherein the plurality of interconnects transmit the electrical signals from between the control circuitry and micropump.

9. The device of claim 1 further comprising a battery for providing power to the control circuitry and micropump.

10. A device configured as a fully autonomous and integrated wearable apparatus for delivery management, the device comprising:
a reservoir for storing the medication for subsequent delivery to a patient;
a needle for delivering the medication to the patient subcutaneously;
a MEMS device configured as a pump for pumping the medication from the reservoir through the needle or as a valve for preventing medication from flowing through device, the MEMS device including a piezoelectric transducer that functions as an actuator;
control circuitry controlling operations of the piezoelectric transducer; and
an interposer integrated with the piezoelectric transducer, the interposer configured as an adapter for mounting the reservoir, the control circuitry and the needle, the interposer including a channel for distributing the medication from the reservoir to the needle and a thin membrane defining a portion of the channel as a chamber,
wherein the piezoelectric transducer is positioned on the thin membrane, thereby deforming the thin membrane as the piezoelectric transducer is actuated.

11. The device of claim 10 wherein the MEMS device includes the thin film membrane that deforms in response to actuation of the piezoelectric transducer thereby increasing or decreasing pressure within the chamber of the channel.

12. An interposer to be used in a device for delivering medication to a patient, the device including a reservoir for storing the medication and a needle for releasing the medication in the patient, the interposer configured to mount the reservoir and needle, the interposer comprising:
a channel for distributing the medication from the reservoir to the needle;
a thin membrane defining a portion of the channel as a chamber for receiving the medication; and
a piezoelectric transducer positioned on the thin membrane that functions as an actuator for moving the thin membrane toward and away from the chamber of the channel.

13. The interposer of claim 12 wherein the piezoelectric transducer and thin membrane function (a) as a pump for displacing or withdrawing medication in the channel or (b) as a valve for preventing medication from moving through the channel.

14. The interposer of claim 12 wherein the medication is insulin.
